(19) United States
(12) Patent Application Publication
(10) Pub. No.: US 2005/0112162 A1
(43) Pub. Date: May 26, 2005

(54) Cosmetic formulation containing sheep's milk and method of making a cosmetic formulation containing sheep's milk

(76) Inventor: Cathy M. Drader, Two Hills (CA)

Correspondence Address: LAWRENCE B. JACKSON;515 SOUTH 5TH, KELSO, WA, 98262, US

(21) Appl. No.: 10/996,410

(22) Filed: Nov. 26, 2004

Related U.S. Application Data
(60) Provisional application No. 60/524,874, filed on Nov. 26, 2003.

(51) Int. Cl. A61K 7/00

(52) U.S. Cl. 424/401

(57) ABSTRACT

There is provided a cosmetic formulation containing sheep's milk and a method for making a cosmetic formulation containing sheep's milk. The cosmetic may be useful for many purposes, including as a nail, skin, face, hair, or general purpose cream. The cosmetic may also be useful for treating nail, skin, face, or other conditions or disorders.

### FIELD OF THE INVENTION

The present invention relates generally to cosmetics. In particular, the invention relates to a cosmetic incorporating sheep's milk and a method for formulating same.

### BACKGROUND OF THE INVENTION

Today, the capacity to improve one's appearance through the use of cosmetics is getting help from modem specialty ingredients and science. Cosmetics are becoming a highly technical business, with the increasing development, availability and sales of products that purportedly mimic the therapeutic effects of pharmaceuticals. Such products have become known as “cosmeceuticals”. This growth has revolutionized the personal care business and enhanced the demand for certain natural ingredients and cosmetic products incorporating such ingredients.

Cosmeceutical products are at the interface of cosmetics, which have cleansing and beautifying properties, and pharmaceuticals, which have curative and healing properties. Cosmeceuticals include products in the areas of skin care, hair care and sun care.

Products which incorporate sheep's dairy products have been found to have beneficial qualities for use as a cosmetic or cosmeceutical, offering beautifying and therapeutic benefits. Beneficial components found in sheep's milk include vitamins A, C, D and E, and minerals including zinc. For example, United Kingdom Patent No. GB 2186484 to Brown entitled “Skin cream preparation” discloses a skin cream made from ewe cream, glycerin, white paraffin, and essential oils, and a method of combining the ingredients involving agitating the ingredients by churning until they are combined.

The technology described in PCT publication no. WO 00/59519 to Kapitz entitled “Active agent and combination of active ingredients for human beings and animals” generally discloses the use of colostrum, the milk secreted during the first few days following birth and characterized by high protein and antibody content, in a skin cream which includes sheep colostrum.

### SUMMARY OF THE INVENTION

In one of its aspects, the cosmetic cream composition according to the invention comprises, in combination: sheep's milk; water; at least one plant-derived oil; and an emulsifier. In another of its aspects, the cosmetic cream composition may further comprise sodium borate and glycerin.

In a further aspect, it may comprise between 6.0 and 80 percent by weight of sheep's milk, and between 14 and 86 percent by weight of water.

In other aspects, the cosmetic cream composition may comprise between 0.0001 and 86 percent by weight of the at least one plant-derived oil and between 0.0001 and 86 percent by weight of the emulsifier.

In yet another aspect, the cosmetic cream composition of the invention may comprise between 1.43 and 3.45 percent by weight of sodium borate in powder form.

In a further aspect, the plant-derived oil may be one of the following oils: vegetable shortening, shea butter, coconut oil, avocado oil, grape seed oil, canola oil, cocoa butter, apricot kernel oil, wheat germ oil, mineral oil, an essential oil, or a fragrance oil, or a mixture of two or more of these oils.

In still another aspect, the cosmetic cream composition may comprise approximately 33 percent by weight of vegetable shortening and approximately 4.0 percent by weight of avocado oil.

In yet a further aspect, the emulsifier may be selected from the group of emulsifiers comprising the following emulsifiers: sodium borate, acrylate copolymer, Crodafos CES, beeswax, cetyl alcohol and stearyl alcohol.

In one embodiment, the invention may comprise between 1.00 and 6.02 percent by weight of beeswax as the emulsifier. In an alternate embodiment, the invention may comprise between 1.50 and 3.69 percent by weight of Crodafos CES as the emulsifier.

In another aspect, the cosmetic cream composition may comprise between 1.14 and 2.01 percent by weight of glycerin.

In still another of its aspects, the cosmetic cream composition may further comprise at least one of the following components: xanthan gum, blue algae extract, licorice root extract, sea parsley extract, dimethicone, microfine zinc oxide, microfine titanium dioxide and citric acid.

In another of its aspects, the invention comprises a method of making a cosmetic composition, comprising the steps of:
a) preparing a first intermediate compound by:
   i) providing water at between 65° C. and 100° C. (149° F. and 212° F.);
   ii) combining the water with a first emulsifier and blending until the first emulsifier is mixed with the water; and
b) cooling the first intermediate compound to between 23.8° C. and 40° C. (75° F. and 104° F.);
c) preparing a second intermediate compound by:
   i) providing a second emulsifier;
   ii) combining the second emulsifier and at least one oil; and
d) adjusting the temperature of the second intermediate compound to between 55° C. and 75° C. (131° F. and 167° F.);
e) forming a cream by combining the first intermediate compound and the second intermediate compound;
f) providing sheep's milk at between 2° C. and 30° C. (35.6° F. and 86° F.) and approximately 30 (86° F.); and
g) combining the sheep's milk and the cream.

In further aspects, the first emulsifier may be sodium borate and the second emulsifier may be melted beeswax.

In another aspect, the oil may be a shortening in liquid form.

In a further aspect, the method of making a cosmetic composition may further comprise cooling the first intermediate compound is to a temperature of between 23.8° C. and 34.0° C. (75.0° F. and 93.2° F.).

In yet a further aspect, the method of making a cosmetic composition may further comprise the additional step, after step (g), of adding one or more of the following: a preservative, a fragrance or a thickener.

### DETAILED DESCRIPTION OF THE PREFERRED EMBODIMENT AND BEST MODE OF THE INVENTION

In general terms, the present invention includes a cosmetic formulation containing sheep's milk and a method for making a cosmetic formulation containing sheep's milk. The cosmetic may be useful for many purposes, including as a nail, skin, face, hair, or general purpose cream. The cosmetic may also be useful for treating nail, skin, face, or other conditions or disorders.

Accordingly, in one aspect, the invention is a cosmetic composition which comprises, in combination:
a) sheep's milk;
b) water;
c) at least one oil; and
d) an emulsifier.

The cosmetic composition may contain between approximately 0.0001 weight (wt.) percent and approximately 80 wt. percent sheep's milk. In the preferred embodiment, the cosmetic composition may contain between approximately 6 wt. percent and approximately 80 wt. percent sheep's milk. The cosmetic composition may contain between approximately 14 wt. percent and approximately 86 wt. percent water. In the preferred embodiment, the cosmetic composition may contain between approximately 0.0001 wt. percent and approximately 85.999 wt. percent of at least one oil. Preferably, the cosmetic composition contains between approximately 0.0001 wt. percent and approximately 86 wt. percent of an emulsifier.

The cosmetic formulation may comprise, in addition, sodium borate and glycerin.

The sheep's milk may be in whole form or skim form with a portion of the cream removed. The milk may pasteurized or not. The sheep's milk may be in liquid, concentrate or powder form. In the preferred embodiment, the cosmetic composition may contain between approximately 0.0001 wt. percent and approximately 80 wt. percent sheep's milk. More preferably, the cosmetic composition contains between approximately 6 wt. percent and approximately 80 wt. percent sheep's milk.

The water used in the formulation may be filtered, for example by reverse osmosis, or may be distilled or deionized. The water may be fresh water, salt water or brine. In the preferred embodiment, the cosmetic formulation may contain between approximately 14 wt. percent and approximately 86 wt. percent fresh water. Preferably the water has been treated or filtered by reverse osmosis.

The sodium borate used in the formulation is preferably in powder form. In the preferred embodiment, the cosmetic may contain between approximately 1.43 wt. percent and approximately 3.45 wt. percent sodium borate.

The at least one oil used in the cosmetic formulation may comprise shortening, shea butter, coconut oil, avocado oil, grape seed oil, canola oil, cocoa butter, apricot kernel oil, wheat germ oil, mineral oil, essential oils (including, for example, lavender, rosemary or sandalwood), fragrance oils, or a mix thereof. The at least one oil may comprise a fatty acid (e.g. avocado oil) or a petroleum oil (e.g. mineral oil), or a mix thereof. By way of example, Table 1 illustrates the approximate composition ranges for several suitable oils for six cosmetic product formulations. Preferably the at least one oil is a mix of vegetable shortening and avocado oil. In the preferred embodiment, the cosmetic may contain approximately 33.3 wt. percent vegetable shortening and approximately 4.01 wt. percent avocado oil.

TABLE 1
Oil Content

| | | Suitable | Preferred |
|---|---|---|---|
| | | Concentration | Concentration |
| Product # | Ingredient | (weight percent) | (weight percent) |
| 1 | Avocado Oil | 0-86 | 2.00-4.01 |
| | Canola Oil | 0-86 | 15.0-33.3 |
| 2 | Avocado Oil | 0-86 | 2.00-4.01 |
| | Cocoa Butter | 0-86 | 2.00-5.00 |
| | Coconut Oil | 0-86 | 4.59 |
| | Shea Butter | 0-86 | 2.00-8.10 |
| 3 | Apricot Oil | 0-86 | 2.50 |
| | Cocoa Butter | 0-86 | 2.00-5.00 |
| 3 | Mineral Oil | 0-86 | 9.29 |
| | Shea Butter | 0-86 | 2.00-8.10 |
| | Wheat Germ Oil | 0-86 | 1.00-1.50 |
| 4 | Cocoa Butter | 0-86 | 2.00-5.00 |
| | Olive Oil | 0-86 | 14.8-15.8 |
| | Shea Butter | 0-86 | 2.00-8.10 |
| | Wheat Germ Oil | 0-86 | 1.00-1.50 |
| 5 | Cocoa Butter | 0-86 | 2.00-5.00 |
| | Olive Oil | 0-86 | 14.8-15.8 |
| | Shea Butter | 0-86 | 2.00-8.10 |
| 6 | Avocado Oil | 0-86 | 2.00-4.01 |
| | Canola Oil | 0-86 | 15.0-33.3 |
| | Shea Butter | 0-86 | 2.00-8.10 |
| | Wheat Germ Oil | 0-86 | 1.00-1.50 |

The emulsifier may be any suitable emulsifier, for example: sodium borate, acrylate copolymer, Crodafos CES®, beeswax, cetyl alcohol, or stearyl alcohol. By way of example, Table 2 illustrates the approximate composition ranges for several suitable emulsifiers. Preferably the emulsifier is beeswax or Crodafos CES or a combination thereof. In the preferred embodiment, the cosmetic composition may contain between approximately 1.00 wt. percent and approximately 6.02 wt. percent beeswax or between approximately 1.50 wt. percent and approximately 3.69 wt. percent Crodafos CES or a combination thereof.

TABLE 2
Emulsifier Content

| | Suitable Concentration | Preferred Concentration |
|---|---|---|
| Ingredient | (weight percent) | (weight percent) |
| Sodium Borate | 0-7 | 0.0-3.5 |
| Acrylate Copolymer | 0-10 | 0.0-7.0 |
| Crodafos CES | 0-10 | 1.5-7.0 |
| Beeswax | 0-10 | 0.0-6.0 |
| Cetyl Alcohol | 0-10 | 0.0-1.0 |
| Stearyl Alcohol | 0-10 | 0.0-2.5 |

The glycerine may be vegetable-derived. In the preferred embodiment, the cosmetic composition may contain between approximately 1.14 wt. percent and approximately 2.01 wt. percent glycerin.

The cosmetic composition may also include a preservative. Preferably, the preservative is Germaben II®. In the preferred embodiment, the cosmetic composition may contain between approximately 1.00 wt. percent and approximately 1.14 wt. percent Germaben II.

The cosmetic composition may also include a fragrance. Preferably, the fragrance is plumaria, lavender, rosemary, or sandalwood.

The cosmetic composition may also include a thickener, such as xanthan gum or Crodafos CES®. The cosmetic composition may contain between approximately 0 wt. percent and approximately 10 wt. percent xanthan gum, but in the preferred embodiment the cosmetic composition may contain between approximately 0 wt. percent and approximately 1 wt. percent xanthan gum.

The cosmetic composition may also include at least one additional additive, such as blue algae extract, licorice root extract, sea parsley extract, dimethicone, microfine zinc oxide, microfine titanium dioxide, citric acid or any combination thereof. By way of example, Table 3 illustrates approximate composition ranges for additional ingredients.

TABLE 3
Optional Ingredient Content

| | Suitable | Preferred |
|---|---|---|
| | Concentration | Concentration |
| Ingredients | (weight percent) | (weight percent) |
| Citric Acid | 0-1 | 0-0.01 |
| Dimethicone | 0.0-2.5 | 0.0-2.5 |
| Microfine Titanium Dioxide | 0-50 | 0-20 |
| Microfine Zinc Oxide | 0-25 | 0.-14 |
| Germaben 11 | 0-1 | 1 |
| Trocopheryl Acetate | 0-10 | 0-5 |
| Blue Algae Extract | 0-2 | 0-1 |
| Ginger Extract | 0-3 | 0-1 |
| Licorice Root extract | 0-20 | 0-10 |
| Myrrh Extract | 0-3 | 0-1 |
| Rosewood Extract | 0-3 | 0-1 |
| Rosehip Extract | 0-3 | 0-1 |
| Sea Parsley extract | 0-1.5 | 0-1.5 |
| Tangerine/Mandarin Extract | 0-3 | 0-1 |
| Ylang Ylang Extract | 0-3 | 0-0.5 |
| Grape Seed Extract | 0-3 | 0.001-1 |
| Fragrance Oil | 0-5 | 0-1.5 |
| Xanthan Gum | 0-10 | 0-1 |

By way of example, Tables 4-7 illustrate various embodiments of the cosmetic composition of the present invention.

TABLE 4
Sample Product Formulation with Whole Sheep's Milk

| Ingredient | Weight percent |
|---|---|
| Distilled water | 42.1 |
| Sodium borate | 2.5 |
| Vegetable shortening | 33.31 |
| Avocado oil | 4.01 |
| Vegetable glycerine | 2.01 |
| Beeswax | 6.02 |
| pasteurized sheep milk | 6.02 |
| Germaben 2 | 1.00 |
| Crodafos CES | 1.50 |
| plumaria | 1.50 |

TABLE 5
Sample Product Formulation with Concentrated Whole Sheep's Milk

| Ingredient | Weight percent |
|---|---|
| Distilled water | 43.1 |
| Sodium borate | 2.5 |
| Vegetable shortening | 33.08 |
| Avocado oil | 4.01 |
| Vegetable glycerine | 2.01 |
| Beeswax | 6.02 |
| pasteurized sheep's milk | 6.02 |
| Germaben II | 1.00 |
| Crodafos CES | 1.51 |
| plumaria | 0.80 |

TABLE 6
Sample Product Formulation with Dried Sheep's Milk

| Ingredient | Weight percent |
|---|---|
| Distilled water | 42.1 |
| Sodium borate | 2.5 |
| Vegetable shortening | 33.31 |
| Avocado oil | 4.01 |
| Vegetable glycerine | 2.01 |
| Beeswax | 6.02 |
| Pasteurized sheep milk | 6.02 |
| Germaben II | 1.00 |
| Crodafos CES | 1.50 |
| plumaria | 1.50 |

TABLE 7
Sample Product Formulation

| Ingredient | Weight percent |
|---|---|
| Water | 57.25 |
| Sodium borate | 3.45 |
| Shea butter | 8.1 |
| Avocado oil | 2.29 |
| Vegetable glycerine | 1.14 |
| Coconut oil | 4.59 |
| Sheep's milk | 13.77 |
| Germaben II | 1.14 |
| Crodafos CES | 3.69 |
| Grape seed oil | 2.29 |
| Cocoa butter | 2.29 |

According to the method of the invention, a method of making a cosmetic composition comprises the steps of:
a) preparing a first intermediate compound by:
   i) providing water at between approximately 65° C. (149° F.) and approximately 100° C. (212° F.);
   ii) combining the water with a first emulsifier, and blending until the first emulsifier is mixed in the water; and
b) cooling the first intermediate compound to between approximately 40° C. (104° F.) and approximately 23.8° C. (75° F.);
c) preparing a second intermediate compound by:
   i) providing a second emulsifier;
   ii) combining the second emulsifier and at least one oil; and
d) adjusting the temperature of the second intermediate compound to between approximately 55° C. (131° F.) and approximately 75° C. (167° F.);
e) forming a cream by combining the first intermediate compound and the second intermediate compound;
f) providing sheep's milk at between approximately 2° C. (35.6° F.) and approximately 30° C. (86° F.); and
g) combining the sheep's milk and the cream.

The first emulsifier may be sodium borate. The second emulsifier may be the same as the first emulsifier or they may differ. The first emulsifier and the second emulsifier may be any emulsifier as listed herein below as an emulsifier. The emulsifier may be a combination of emulsifiers.

The water used in the method may be filtered, for example by reverse osmosis or otherwise or may be distilled or deionized. The emulsifier used in the method may comprise beeswax. If the emulsifier comprises beeswax, it may be melted over a low heat before it is added to the second intermediate compound.

The at least one oil used in the method may comprise shortening. If the shortening is in solid or semi-solid form, it may be melted to be provided in liquefied form. Preferably it is melted over a low heat before being added to the second intermediate compound in liquid form.

Preferably the temperature of the first intermediate compound is adjusted to between approximately 34° C. (93.2° F.) and approximately 23.8° C. (75° F.).

According to the method of the invention, the cream is formed by adding the first intermediate compound to the second intermediate compound, and blending or mixing them together. In the preferred embodiment, the first and second intermediate compounds are combined at a rate sufficiently slow to substantially avoid separation of the oil and water.

The sheep's milk used in the method may be pasteurized. The sheep's milk may be thawed and refrozen as desired to allow for safer or simpler transport. In the preferred embodiment, the sheep's milk may be prepared by the steps of pasteurizing and freezing, and then is provided at a temperature in the range specified above. In the preferred embodiment, the sheep's milk is added to the cream and blended until mixed. In the preferred embodiment the sheep's milk is provided at approximately room temperature.

If powdered sheep's milk is used, it is prepared according to the supplier's instructions, typically by the addition of a specified amount of water and then mixing. The reconstituted sheep's milk can then be used in the same way as sheep's milk previously disclosed.

If concentrated sheep's milk is used, then it is reconstituted according to the supplier's instructions, typically by the addition of a specified amount of water and then mixing. The reconstituted milk can then used in the same way as sheep's milk previously disclosed.

Optional ingredients may be added to the cosmetic after the sheep's milk is mixed. Optional ingredients include a preservative, preferably Germaben® II, or a fragrance such as plumeria or any of the additives or thickeners previously mentioned.

It will be readily seen by those skilled in the art that various modifications of the present invention may be devised without departing from the essential concept of the invention, and all such modifications are intended to be included in the scope of the claims appended hereto.

1. A cosmetic cream composition comprising, in combination:
sheep's milk;
water;
at least one plant-derived oil; and
an emulsifier.

2. The cosmetic cream composition of claim 1, further comprising:
sodium borate; and
glycerin.

3. The cosmetic cream composition of claim 1, comprising between 6.0 and 80 percent by weight of sheep's milk.

4. The cosmetic cream composition of claim 1, comprising between 14 and 86 percent by weight of water.

5. The cosmetic cream composition of claim 1, comprising between 0.0001 and 86 percent by weight of the at least one plant-derived oil.

6. The cosmetic cream composition of claim 1, comprising between 0.0001 and 86 percent by weight of the emulsifier.

7. The cosmetic cream composition of claim 7, comprising between 1.43 and 3.45 percent by weight of sodium borate in powder form.

8. The cosmetic cream composition of claim 1, wherein the at least one plant-derived oil is one of the following oils: vegetable shortening, shea butter, coconut oil, avocado oil, grape seed oil, canola oil, cocoa butter, apricot kernel oil, wheat germ oil, mineral oil, an essential oil, or a fragrance oil, or a mixture of two or more of these oils.

9. The cosmetic cream composition of claim 8, comprising approximately 33.3 percent by weight of vegetable shortening and 4.01 percent by weight of avocado oil.

10. The cosmetic cream composition of claim 1, wherein the emulsifier is selected from the group of emulsifiers comprising the following emulsifiers: sodium borate, acrylate copolymer, Crodafos CES, beeswax, cetyl alcohol and stearyl alcohol.

11. The cosmetic cream composition of claim 1, comprising between 1.00 and 6.02 percent by weight of beeswax as the emulsifier.

12. The cosmetic cream composition of claim 1, comprising between 1.50 and 3.69 percent by weight of Crodafos CES as the emulsifier.

13. The cosmetic cream composition of claim 7, comprising between 1.14 and 2.01 percent by weight of glycerin.

14. The cosmetic cream composition of claim 1, further comprising at least one of the following components: xanthan gum, blue algae extract, licorice root extract, sea parsley extract, dimethicone, microfine zinc oxide, microfine titanium dioxide and citric acid.

15. A method of making a cosmetic composition, said method comprising the steps of:
a) preparing a first intermediate compound by:
i) providing water at between 65° C. and 100° C. (149° F. and 212° F.);
ii) combining the water with a first emulsifier and blending until the first emulsifier is mixed with the water; and
b) cooling the first intermediate compound to between 23.8° C. and 40° C. (75° F. and 104° F.);
c) preparing a second intermediate compound by:
i) providing a second emulsifier;
ii) combining the second emulsifier and at least one oil; and
d) adjusting the temperature of the second intermediate compound to between 55° C. and 75° C. (131° F. and 167° F.);
e) forming a cream by combining the first intermediate compound and the second intermediate compound;
f) providing sheep's milk at between 2° C. and 30° C. (35.6° F. and 86° F.) and approximately 30 (86° F.); and
g) combining the sheep's milk and the cream.

16. The method of making a cosmetic composition of claim 15, wherein the first emulsifier is sodium borate.

17. The method of making a cosmetic composition of claim 16, wherein the second emulsifier comprises melted beeswax.

18. The method of making a cosmetic composition of claim 15, wherein the at least one oil is a shortening in liquid form.

19. The method of making a cosmetic composition of claim 15, wherein the first intermediate compound is cooled to a temperature of between 23.8° C. and 34.0° C. (75.0° F. and 93.2° F.).

20. The method of making a cosmetic composition of claim 15, further comprising the additional step, after step (g), of adding one or more of the following: a preservative, a fragrance or a thickener.
